(19) United States
(12) Patent Application Publication
(10) Pub. No.: US 2008/0076049 A1
(43) Pub. Date: Mar. 27, 2008

(54) ARYLAMINE PROCESSES

(75) Inventors: Marko D. Saban, Etobicoke (CA); Leanne Dawn Murphy, Etobicoke (CA); Thomas Edward Enright, Tottenham (CA)

Correspondence Address: OLIFF & BERRIDGE, PLC., P.O. BOX 320850, ALEXANDRIA, VA, 22320-4850, US

(73) Assignee: XEROX CORPORATION, Stamford, CT (US)

(21) Appl. No.: 11/535,301

(22) Filed: Sep. 26, 2006

(51) Int. Cl. C07C 229/52; G03G 15/02

(52) U.S. Cl. 430/58.05; 562/433

(57) ABSTRACT

Continuous and batch processes for selectively hydrogenating double and/or triple bonds in organic molecules include providing at least one organic molecule containing a double and/or triple bond, providing at least one hydrogen donor molecule, and hydrogenating the double and/or triple bond in the presence of at least one catalyst. Continuous and batch processes for preparing arylamine molecules include selectively hydrogenating double bonds in arylamine compounds by providing at least one organic molecule containing a multiple bond, providing at least one hydrogen donor molecule, and hydrogenating the multiple bond in the presence of at least one catalyst. The continuous and batch processes provide efficient, cost-effective and safe methods for conducting selective hydrogenation reactions in the synthesis of organic molecules, such as charge-transport molecules.

### TECHNICAL FIELD

This disclosure is generally directed to processes for the synthesis of arylamine compounds, and to the use of such compounds in electrophotographic imaging members. In particular, this disclosure provides processes for the selective hydrogenation of unsaturated arylamine compounds.

### RELATED APPLICATIONS

Commonly assigned U.S. patent application Ser. No. 11/380,339, filed Apr. 26, 2006, describes a process for selectively hydrogenating double and/or triple bonds in organic molecules, comprising: providing one or more acceptor molecules that contains one or more alkyl groups having one or more double bonds and/or one or more triple bonds; providing one or more hydrogen donor molecules; and hydrogenating said double bonds and/or said triple bonds of said organic molecules in the presence of one or more catalysts.

Commonly assigned U.S. patent application Ser. No. 11/263,671, filed Nov. 1, 2005, describes a process for the preparation of a tertiary arylamine compound, comprising reacting an aryl halide and an arylamine in an ionic liquid in the presence of a catalyst.

Commonly assigned U.S. patent application Ser. No. 11/094,683, filed Mar. 31, 2005, describes a process for forming an anhydrous alkali earth salt of a dicarboxylic acid of an arylamine compound, comprising reacting a dicarboxylic acid of an arylamine compound with an anhydrous alkali earth salt. The application also discloses a process for forming a siloxane-containing hole-transport molecule, comprising reacting a dicarboxylic acid of an arylamine compound with an anhydrous alkali earth salt to form an anhydrous dicarboxylic acid salt of the arylamine compound; and reacting the anhydrous dicarboxylic acid salt of the arylamine compound with a siloxane-containing compound.

Commonly assigned U.S. patent application Ser. No. 11/034,713, filed Jan. 14, 2005, describes an electrophotographic photoreceptor comprising a charge-generating layer, a charge-transport layer, and an overcoat layer comprised of a crosslinked siloxane composite composition comprising at least one siloxane-containing compound and metal oxide particles.

Commonly assigned U.S. patent application Ser. No. 10/998,585, filed Nov. 30, 2004, describes a silicon-containing layer for electrophotographic photoreceptors comprising: one or more siloxane-containing compound; and one or more siloxane-containing antioxidant, wherein the siloxane-containing antioxidant is at least one member selected from the group consisting of hindered-phenol antioxidants, hindered-amine antioxidants, thioether antioxidants and phosphite antioxidants.

Commonly assigned U.S. patent application Ser. No. 10/992,690, filed Nov. 2, 2004, describes a process for forming a tertiary arylamine compound, comprising reacting an arylbromide and an arylamine. For example, the application describes a process for forming N,N-diphenyl-4-aminobiphenyl, comprising reacting 4-bromobiphenyl and diphenylamine in the presence of a palladium-ligated catalyst.

Commonly assigned U.S. patent application Ser. No. 10/992,687, filed Nov. 22, 2004, describes a process forming a 4-aminobiphenyl derivative arylamine compound, comprising: (i) providing a first disubstituted 4-aminobiphenyl compound; (ii) optionally formylating the first disubstituted 4-aminobiphenyl compound to form a bisformyl substituted compound, where the first disubstituted 4-aminobiphenyl compound is not a bisformyl substituted compound; (iii) acidifying the bisformyl substituted compound to convert formyl functional groups into acid functional groups to form an acidified compound; and (iv) hydrogenating the acidified compound to saturate at least one unsaturated double bonds in the acidified compound, wherein there is provided a second disubstituted 4-aminobiphenyl compound.

Commonly assigned U.S. patent application Ser. No. 10/992,658, filed Nov. 22, 2004, describes a process for forming a 4-aminobiphenyl derivative arylamine compound, comprising: (i) providing an iodinated organic compound; (ii) substituting the iodinated organic compound at carboxylic acid groups thereof to provide ester protecting groups; (iii) conducting an Ullman condensation reaction to convert the product of step (ii) into an arylamine compound; and (iv) conducting a Suzuki coupling reaction to add an additional phenyl group to the arylamine compound in the 4-position relative to the nitrogen, to provide the 4-aminobiphenyl derivative arylamine compound.

Commonly assigned U.S. patent application Ser. No. 10/709,193, filed Apr. 20, 2004, which published as U.S. Patent Application Publication No. 2005-0234272 on Oct. 20, 2005, describes a process for preparing an aryl iodide compound, comprising: reacting an aryl halide compound with a metal iodide, a metal catalyst and a catalyst coordinating ligand in at least one solvent to form an aryl iodide; and purifying the aryl iodide; wherein the solvent is heated to reflux during the reacting; wherein an aryl iodide yield of at least about 75% is obtained; and wherein the aryl iodide has a purity of at least 90%.

Commonly assigned U.S. patent application Ser. No. 10/600,110, filed Jun. 20, 2003, which published as U.S. Patent Application Publication No. 2004-0260026 on Dec. 23, 2004, describes a process comprising: heating to a first polymerization temperature a first mixture comprising a free radical polymerizable monomer, a free radical initiator, and a stable free radical compound to polymerize only a portion of the monomer, resulting in a prepolymer composition; shearing a second mixture including the prepolymer composition, a continuous phase liquid, and a stabilizing compound to create a miniemulsion; and flowing the miniemulsion within a polymerization reactor and heating the miniemulsion at a second polarization temperature to form polymeric particles wherein the formation of the polymeric particles occurs while the miniemulsion flows within the reactor.

The appropriate components and process aspects of each of the foregoing, such as the arylamine precursor materials and electrophotographic imaging members, may be selected for the present disclosure in embodiments thereof. The entire disclosures of the above-mentioned applications are totally incorporated herein by reference.

### REFERENCES

JP-A-63-65449 (the term “JP-A” as used herein means an “unexamined published Japanese patent application”), discloses an electrophotographic photoreceptor in which fine silicone particles are added to a photosensitive layer, and also discloses that such addition of the fine silicone particles imparts lubricity to a surface of the photoreceptor.

Further, in forming a photosensitive layer, a method has been proposed in which a charge-transport substance is dispersed in a binder polymer or a polymer precursor thereof, and then the binder polymer or the polymer precursor thereof is cured. JP-B-5-47104 (the term “JP-B” as used herein means an “examined Japanese patent publication”) and JP-B-60-22347, disclose electrophotographic photoreceptors using silicone materials as the binder polymers or the polymer precursors thereof.

Furthermore, in order to improve mechanical strength of the electrophotographic photoreceptor, a protective layer is formed on the surface of the photosensitive layer in some cases. A cross-linkable resin is used as a material for the protective layer in many cases. However, the protective layer formed by the cross-linkable resin acts as an insulating layer, which impairs the photoelectric characteristics of the photoreceptor. For this reason, a method of dispersing a fine conductive metal oxide powder (JP-A-57-128344) or a charge-transport substance (JP-A-4-15659) in the protective layer and a method of reacting a charge-transport substance having a reactive functional group with a thermoplastic resin to form the protective layer have been proposed.

However, even the above-mentioned conventional electrophotographic photoreceptors are not necessarily sufficient in electrophotographic characteristics and durability, particularly when used in combination with a charger of the contact-charging system (contact charger) or a cleaning apparatus, such as a cleaning blade.

Further, when a photoreceptor is used in combination with a contact charger and a toner obtained by chemical polymerization (polymerization toner), a surface of the photoreceptor may become stained with a discharge product produced in contact charging or with polymerization toner that remains after a transport step. This staining can deteriorate image quality in some cases. Still further, use of a cleaning blade to remove discharge product or remaining toner adhered to the photoreceptor surface increases friction and abrasion between the surface of the photoreceptor and the cleaning blade, resulting in a tendency to cause damage to the surface of the photoreceptor, breakage of the blade or turning up of the blade.

Furthermore, in producing a photoreceptor, in addition to improvement in electrophotographic characteristics and durability, reducing production costs becomes an important problem. However, conventional electrophotographic photoreceptors also may have problems relating to coating defects such as orange-peel appearances and hard spots.

The use of silicon-containing compounds in photoreceptor layers, including in photosensitive and protective layers, has been shown to increase the mechanical lifetime of electrophotographic photoreceptors, under charging conditions and scorotron-charging conditions. For example, U.S. Patent Application Publication US 2004/0086794 to Yamada et al. discloses a photoreceptor having improved mechanical strength and stain resistance.

However, the above-mentioned conventional electrophotographic photoreceptor is not necessarily sufficient in electrophotographic characteristics and durability, particularly when such a photoreceptor is used in an environment of high heat and humidity.

Photoreceptors having low wear rates, such as those described in Yamada, also have low refresh rates. The low wear and refresh rates are a primary cause of image-deletion errors, particularly under conditions of high humidity and high temperature. U.S. Pat. No. 6,730,448 B2 to Yoshino et al. addresses this issue, disclosing photoreceptors having some improvement in image quality, fixing ability, even in an environment of high heat and humidity. However, there still remains a need for electrophotographic photoreceptors having high mechanical strength and improved electrophotographic characteristics and improved image-deletion characteristics even under conditions of high temperature and high humidity.

Over the past several years, Buchwald et al. (MIT) and Hartwig et al. (Yale) have both reported on the general versatility of palladium-based catalysts for the formation of nitrogen-carbon bonds. Their work has focused on the arylation of alkylamine and alkylamides, but they have reported the use of palladium-based catalysts for arylamine synthesis starting from an aryl bromide or an aryl chloride. See Harris, M. C. et al; One-Pot Synthesis of Unsymmetrical Triarylamines from Aniline Precursors, J. Org. Chem., 65, 5327 (2000).

Brieger, G. et al., Catalytic Transfer Hydrogenation, Chemical Reviews 74(5) 567 (1974), and Johnstone, R. A. W. et al., Heterogeneous Catalytic Transfer Hydrogenation and its Relation to Other Methods for Reduction of Organic Compound, Chem. Rev. 85, 129 (1985), describe transfer hydrogenation-dehydrogenation reactions in which hydrogen is removed from donor molecules and selectively added to acceptor molecules in the presence of a catalysts.

Narisada, M. et al., Selective Reduction of Aryl Halides and α,β-Unsaturated Esters with Sodium Borohydride-Cuprous Chloride in Methanol and its Application of Deuterium Labeling, J. Org. Chem., 54, 5308 (1989); Satoh, T. et al., Reduction of Organic Compounds with NaBH₄-Transition Metal Salt Systems. IV. Selective Hydrogenation of Olefins in Unsaturated Esters, Chem. & Pharm. Bull., 19(4), 817 (1971); Sim, T. B. et al., Selective Reduction of α,β-Unsaturated Acid Derivatives using Borohydride Exchange Resin-CuSO₄ in Methanol, Synthesis Letters, 7, 726 (1995); and Yoon. M. Selective Reduction of Organic Compounds with Aluminum and Boron Hydrides, Pure & Appl. Chem. 68 (4), 843 (1996), describe metal hydride-transition metal salt catalytic systems that selectively reduce carbon-carbon double bonds.

U.S. Pat. No. 6,683,025 B2 to Amendola et al. describes a process for producing a supported hydrogen generation catalyst comprising: contacting an anionic exchange resin substrate with a solution comprising an anionic complex of a transition metal ion, said complex represented by the formula (M^{y}+X₆)^{(y-6)}, wherein M is a transition metal, y is the valence of the transition metal, and X is an anion with a single negative charge, thereby effecting an exchange reaction whereby the anion associated with the exchange resin is substituted with the anion of said transition metal complex, wherein said solution is obtained by dissolving a transition metal salt in an acid to form said complex, further wherein the anion of said acid corresponds to the anion of said transition metal salt; and contacting the resulting anionic exchange resin substrate with a reducing agent.

The disclosures of each of the foregoing patents and publications, and the disclosures of any patents and publications cited below, are hereby totally incorporated by reference. The appropriate components and process aspects of the each of the cited patents and publications may also be selected for the present compositions and processes in embodiments thereof.

### BACKGROUND

In electrophotography, an electrophotographic substrate containing a photoconductive insulating layer on a conductive layer is imaged by first uniformly electrostatically charging a surface of the substrate. The substrate is then exposed to a pattern of activating electromagnetic radiation, such as, for example, light. The electromagnetic radiation selectively dissipates charge in illuminated areas of the photoconductive insulating layer while leaving behind an electrostatic latent image in non-illuminated areas of the photoconductive insulating layer. This electrostatic latent image is then developed to form a visible image by depositing finely divided electroscopic marking particles on the surface of the photoconductive insulating layer. The resulting visible image is then transferred from the electrophotographic substrate to a necessary member, such as, for example, an intermediate-transfer member or a print substrate, such as paper. This image developing process can be repeated as many times as necessary with reusable photoconductive insulating layers.

In image-forming apparatus such as copiers, printers and facsimiles, electrophotographic systems in which charging, exposure, development, transfer, etc. are carried out using electrophotographic photoreceptors have been widely employed. In such image-forming apparatus, there are ever-increasing demands for speeding up of image-formation processes, improvement in image quality, miniaturization and prolonged life of the apparatus, reduction in production cost and running cost, etc. Further, with recent advances in computers and communication technology, digital systems and color-image output systems have been applied also to the image-forming apparatus.

Electrophotographic imaging members (photoreceptors) are known. Electrophotographic imaging members are commonly used in electrophotographic processes having either flexible-belt or rigid-drum configurations. These electrophotographic imaging members sometimes comprise a photoconductive layer including a single layer or composite layers. These electrophotographic imaging members take many different forms. For example, layered photo-responsive imaging members are known in the art. U.S. Pat. No. 4,265,990 to Stolka et al. describes a layered photoreceptor having separate photogenerating and charge-transport layers. The photogenerating layer disclosed in Stolka is capable of photogenerating holes and injecting the photogenerated holes into the charge-transport layer. Thus, in the photoreceptors of Stolka, the photogenerating material generates electrons and holes when subjected to light.

More advanced photoconductive photoreceptors containing highly specialized component layers are also known. For example, a multi-layered photoreceptor employed in electrophotographic imaging systems sometimes includes one or more of a substrate, an undercoating layer, an intermediate layer, an optional hole- or charge-blocking layer, a charge-generating layer (including a photogenerating material in a binder) over an undercoating layer and/or a blocking layer, and a charge-transport layer (including a charge-transport material in a binder). Additional layers such as one or more overcoat layer or layers are also sometimes included.

In view of such a background, improvement in electrophotographic properties and durability, miniaturization, reduction in cost, and the like, in electrophotographic photoreceptors have been studied, and electrophotographic photoreceptors using various materials have been proposed.

Production of a number of arylamine compounds, such as arylamine compounds that are useful as charge-transport compounds in electrostatographic imaging devices and processes, often involves synthesis of intermediate materials, some of which generally are costly and/or time-consuming to produce, and some of which involve a multi-step process. One such intermediate product is the arylamine N,N-di(4-propanoic acid)-4-aminobiphenyl, which is itself useful as a charge-transport compound in electrostatographic imaging devices and processes. Even production of this intermediate compound currently involves a long, costly process.

Currently, arylamine-derivative hole-transporting molecules are prepared by a process that includes reducing or hydrogenating a double bond using compressed hydrogen gas. For example, N,N-di(4-propanoic acid)-4-aminobiphenyl has been produced by selectively reducing N,N-di(4-propenoic acid)-4-aminobiphenyl using compressed hydrogen (H₂) gas. While known and useful on a small, laboratory scale, this method is not conducive to large-scale production of arylamine-derivative hole-transporting molecules, because it is costly and poses safety concerns.

First, hydrogen gas is a highly diffusible and highly combustible gas. The safety requirements for equipment and facilities for using hydrogen gas are strict, and altering equipment and facilities to meet or exceed the safety requirements for larger scale hydrogenation reactions could be very costly, particularly in light of the small volume necessary for the preparation of arylamine molecules.

Second, the efficiency of conventional hydrogenation reactions using compressed hydrogen gas depends on converting large amounts of the hydrogen gas to the liquid phase. In order to increase efficiency, elevated pressure and temperature, which would require specialized mixing equipment, would be necessary, and would, in turn, increase production costs.

Accordingly, improved processes providing safe, cost-effective and efficient methods for selective hydrogenation are desired for producing arylamines, such as N,N-di(4-propanoic acid)-4-aminobiphenyl, and similar compounds.

### SUMMARY

The present disclosure addresses these and other needs, by providing continuous and batch processes for the selective hydrogenation of unsaturated organic molecules. In particular, provided are continuous and batch processes that include selective hydrogenation of organic molecules by catalytic transfer hydrogenation processes to avoid problems and costs associated with reduction by compressed hydrogen gas. Such processes can achieve safer synthetic processes without requiring costly upgrades to equipment and facilities, and can enable selective hydrogenation to be performed in standard equipment.

Exemplary methods include processes for selectively hydrogenating double and/or triple bonds in organic molecules, comprising: providing one or more acceptor molecules that contains one or more double bonds and/or one or more triple bonds; providing one or more hydrogen donor molecules; and hydrogenating the double bonds and/or the triple bonds of the organic molecules in the presence of one or more catalysts.

Additional exemplary methods include processes for preparing arylamine molecules, comprising: selectively hydrogenating double bonds in arylamine compounds, wherein selectively hydrogenating double bonds comprises: providing one or more arylamine compounds that contains one or more double bonds and/or one or more triple bonds; providing one or more hydrogen donor molecules; and hydrogenating the double bonds and/or the triple bonds of the arylamine compounds in the presence of one or more catalysts.

In addition, embodiments include electrophotographic imaging members comprising: a substrate; a charge-generating layer; a charge-transport layer; and optionally an overcoating layer; wherein the charge-transporting layer includes one or more charge-transporting molecules prepared by a process that comprises: providing one or more acceptor molecules that contains one or more double bonds and/or one or more triple bonds; providing one or more hydrogen donor molecules; and hydrogenating the double bonds and/or the triple bonds of the organic molecules in the presence of one or more catalysts.

These and other features and advantages of various embodiments of materials, devices, systems and/or methods are described in or are apparent from, the following detailed description.

### BRIEF DESCRIPTION OF THE DRAWINGS

These and other advantages and features of this disclosure will be apparent from the following, especially when considered with the accompanying drawings, in which:

FIG. 1 is a schematic view showing an embodiment of a continuous hydrogenation reaction system, including a fixed catalyst bed, according to an embodiment of the disclosure.

FIG. 2 is a reaction scheme showing a generalized hydrogenation of the disclosure.

FIG. 3 is a reaction scheme showing a generalized hydrogenation of the disclosure.

FIG. 4 is a schematic cross sectional view showing an embodiment of an electrophotographic photoreceptor of the disclosure.

FIG. 5 is a schematic view showing an embodiment of an image-forming apparatus of the disclosure.

FIG. 6 is a schematic view showing another embodiment of an image-forming apparatus of the disclosure.

### DETAILED DESCRIPTION OF EMBODIMENTS

This disclosure is not limited to particular embodiments described herein, and some components and processes may be varied by one of skill, based on this disclosure. The terminology used herein is for the purpose of describing particular embodiments only, and is not intended to be limiting.

In this specification and the claims that follow, singular forms such as “a,” “an,” and “the” include plural forms unless the content clearly dictates otherwise. In addition, reference may be made to a number of terms that shall be defined as follows:

The term “organic molecule” refers, for example, to any molecule that is made up predominantly of carbon and hydrogen, such as, for example, alkanes and arylamines. The term “heteroatom” refers, for example, to any atom other than carbon and hydrogen. Typical heteroatoms included in organic molecules include oxygen, nitrogen, sulfur and the like.

The term “saturated” refers, for example, to compounds containing only single bonds. The term “unsaturated” refers, for example, to compounds that contain one or more double bonds and/or one or more triple bonds.

The terms “hydrocarbon” and “alkane” refer, for example, to branched and unbranched organic molecules having the general formula CₙH₂ₙ₊₂, in which n is a number of 1 or more, such as of from about 1 to about 60. Exemplary alkanes include methane, ethane, n-propane, isopropane, n-butane, isobutane, tert-butane, octane, decane, tetradecane, hexadecane, eicosane, tetracosane and the like. Alkanes may be substituted by replacing hydrogen atoms with one or more functional groups.

The term “aliphatic” refers, for example, to straight-chain molecules, and may be used to describe acyclic, unbranched alkanes.

The term “long-chain” refers, for example, to hydrocarbon chains in which n is a number of from about 8 to about 60, such as from about 20 to about 45 or from about 30 to about 40. The term “short-chain” refers, for example, to hydrocarbon chains in which n is a number of from about 1 to about 7, such as from about 2 to about 5 or from about 3 to about 4.

The term “aromatic” refers, for example, to an organic molecule or radical in which some of the bonding electrons are delocalized or shared among several atoms within the molecule and not localized in the vicinity of the atoms involved in the bonding. Aromatic compounds may include heteroatoms in the molecules, and may include one or more cyclic or ring, systems that may include one or more fused aromatic rings. Examples of aromatic compounds include, for example, benzene (C₆H₆), naphthalene (C₁₀H₈), anthracene (C₁₄H₁₀), pyridine (C₅H₅N) and the like. Optionally, these aromatic compounds may be substituted with one or more independently selected substituents, including alkyl, alkenyl, alkoxy, aryl, hydroxyl and nitro groups.

The term “aryl” refers, for example, to an organic group derived from an aromatic compound and having the same general structure as the aromatic compound. Examples of aromatic compounds include, for example, phenyl (C₆H₅), benzyl (C₇H₇), naphthyl (C₁₀H₇), anthracyl (C₁₄H₉), pyridinyl (C₅H₄N) and the like. Optionally, these aromatic groups may be substituted with one or more independently selected substituents, including alkyl alkenyl, alkoxy, aryl, hydroxyl and nitro groups.

The term “arylamine” refers, for example, to moieties containing both aryl and amine groups. Exemplary arylamine groups have the stricture Ar—NRR′, in which Ar represents an aryl group and R and R′ are groups that may be independently selected from hydrogen and substituted and unsubstituted alkyl, alkenyl, aryl and other suitable functional groups. The term “triarylamine” refers, for example, to arylamine compounds having the general structure NArAr′Ar″, in which Ar, Ar′ and Ar″ represent independently selected aryl groups.

The term “functional group” refers, for example, to a group of atoms arranged in a way that determines the chemical properties of the group and the molecule to which it is attached. The term “functional group” includes: (1) a “charged functional group” that contains a positive or negative charge; and (2) a “neutral functional group” that is neutral (not charged) but can be induced by ionization to result in a positive or negative charge. Ionization can be induced by for example heating, electrical potential, changing pH, and the like. For example, a carboxylic acid as the neutral functional group can be taken to a basic pH to generate the carboxylate charged functional group. The neutral functional group can be, for example, carboxylic acids, sulphonic acids, phosphates, amines and the like. The charged functional group can be, for example, carboxylates, sulfonates, phosphates, quaternary amines, and the like. The substituents on the functional group can be aromatic, aliphatic or combinations thereof.

The term “derivative” refers, for example, to compounds that are derived from another compound and maintain the same general structure as the compound from which they are derived. For example, saturated alcohols and saturated amines are derivatives of alkanes.

The terms “standard temperature” and “standard pressure” refer, for example, to the standard conditions used as a basis where properties vary with temperature and/or pressure. Standard temperature is 0° C.; standard pressure is 101,325 Pa or 760.0 mmHg. The term “room temperature” refers, for example, to temperatures in a range of from about 20° C. to about 25° C.

The terms “selective” and “selectively” refer, for example, to reactions in which the reaction occurs at only one reaction site of multiple possible reaction sites where such a reaction could theoretically occur. For example, a selective hydrogenation reaction of a propenoic acid compound may add hydrogen across only the carbon-carbon double bond, and not across the carbon-oxygen double bond, to form a propanoic acid compound.

The terms “continuous” (as used in “continuous reaction,” “continuous mode” and “continuous amount”) and “batch” (as used in “batch reaction,” “batch mode” and “batch amount”) are used in their ordinary sense in the chemical arts to differentiate the two basic types of manufacturing processes.

The terms “optional” and “optionally” refer, for example, to instances in which subsequently described circumstance may or may not occur, and include instances in which the circumstance occurs and instances in which the circumstance does not occur.

The terms “one or more” and “at least one” refer, for example, to instances in which one of the subsequently described circumstances occurs, and to instances in which more than one of the subsequently described circumstances occurs. Similarly, the terms “two or more” and “at least two” refer, for example to instances in which two of the subsequently described circumstances occurs, and to instances in which more than two of the subsequently described circumstances occurs.

In a catalytic transfer-hydrogenation reaction, hydrogen is transferred from a donor molecule to an acceptor molecule. The release of hydrogen from the donor requires a catalyst that transfers the hydrogen to the acceptor. That is, the catalyst abstracts the hydrogen from the donor molecule. This is illustrated below as exemplary reaction (1). In reaction (1), hydrogen is removed from ammonium formate, shown here as formyl and ammonium ions. The products of this reaction are CO₂, NH₃ and H₂, all of which are non-toxic and can be easily removed from the system.

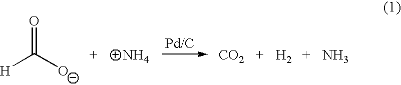

Once hydrogen has been removed from the donor molecule, it may be transferred to an acceptor molecule. That is, hydrogen ions abstracted from the donor molecule react to reduce a multiple bond in the acceptor molecule, resulting in cis-addition of hydrogen across a double or triple bond in the acceptor molecule.

The acceptor molecule in embodiments of catalytic transfer hydrogenation processes disclosed herein may be any organic molecule that contains one or more alkyl groups having one or more double bond and/or one or more triple bond. In embodiments, acceptor molecules may be any suitable arylamine, depending on the desired final product, that includes one or more double bond and/or one or more triple bond to be reduced. For example, the arylamine may correspond to the exemplary triarylamine (A) below. Triarylamine (A) includes substituents R¹-R¹⁵, which can be the same or different, can be suitably selected to represent hydrogen, a halogen, an alkyl group having for example from 1 to about 20 carbon atoms, an aryl group optionally substituted by one or more alkyl groups, an alkyl group containing a heteroatom, an aryl group containing a heteroatom and optionally substituted by one or more alkyl groups, and the like, as long as at least one of R¹-R¹⁵ includes a multiple bond. In embodiments, the arylamine is a diphenylamine derivative.

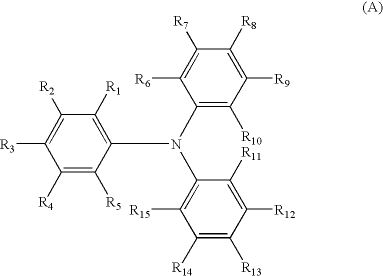

In embodiments, one or more of R⁶-R¹⁵ of the arylamine may include an acrylic acid group, which may be selectively hydrogenated by catalytic transfer hydrogenation. Suitable acrylic acid-containing arylamines may include N,N-di(4-propenoic acid)-4-aminobiphenyl and homologous molecules. The organic acceptor molecule of embodiments may be one organic acceptor molecule as described above or may be a mixture of two or more such organic acceptor molecules.

In embodiments, the acceptor molecule may be reacted in any suitable amount to obtain the desired product.

In embodiments, the donor molecule of catalytic transfer hydrogenation processes disclosed herein may be an organic donor molecule. Suitable donor molecules include organic compounds that may be oxidized under mild conditions. The term “mild conditions” refers, for example, to conditions having a temperature of from about 0° C. to about 100° C., such as from about 20° C. to about 90° C. or from about 50° C. to about 70° C.; and standard pressure.

The donor molecules are not otherwise particularly limited. Non-limiting examples of suitable donor molecules include hydrazine; formic acid and formates, such as ammonium formate; substituted and unsubstituted cyclohexenes; substituted and unsubstituted octalins; substituted and unsubstituted tetralins; substituted and unsubstituted pinenes; substituted and unsubstituted carenes; substituted and unsubstituted phellandrenes; substituted and unsubstituted terpinolenes; substituted and unsubstituted menthenes; substituted and unsubstituted cadalene; substituted and unsubstituted pulegones; substituted and unsubstituted selinenes; and alcohols, such as methanol, ethanol, 2-propanol, octanol, diethylcarbinol, cyclohexanol, benzyl alcohol, phenylethanols, cyclohexylphenols and the like. See Brieger, at 568. The donor molecule of embodiments may be one such donor molecule or may be a mixture of two or more such donor molecules.

In embodiments, the donor molecule may be reacted in amounts from about 1 to about 10 molar equivalents, such as from about 1 to about 4 or from about 1 to about 2.5 molar equivalents weight, per molar equivalent of acceptor molecule.

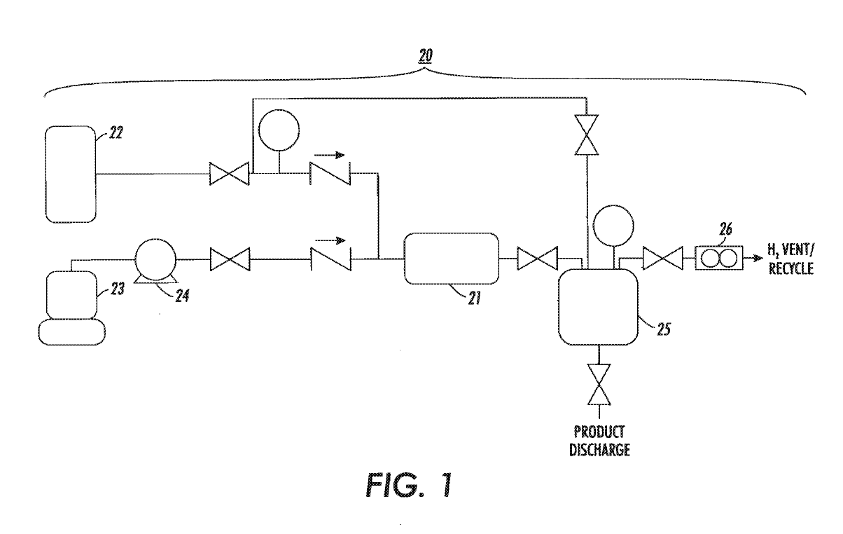

The acceptor and donor molecules of embodiments are provided, in the form of a mixed feed solution, to a continuous reaction system. Suitable reaction systems are known in the chemical arts, and include, for example, those known in the petrochemical arts and the emulsion-polymerization arts. For example, a suitable reaction system for embodiments may include a continuous tubular reactor that may contain a fixed catalyst bed. Such as continuous tubular reactor system 20, as shown in FIG. 1, may include a packed-bed reactor 21 that contains a hydrogenation catalyst. The packed-bed reactor 21 may be fed reactants from hydrogen tank 22 and from liquid reagent tank 23 by a pump 24. The packed-bed reactor may be contained or immersed in a bath, which may be temperature controlled, so that the reaction temperature may be controlled. The reaction mixture is fed from the packed bed reactor 21 to a separation unit 25, from which hydrogenated product is obtained and hydrogen is vented via hydrogen flowmeter 26. An advantage of such continuous reactor systems is that the continuous tubular reactor apparatus can be contained and operated in a laboratory fume hood.

In other embodiments, a fluidized-bed system, in which reactants flow through a fluidized bed in which the catalyst is confined, may be used as the reaction system.

The continuous reaction system of embodiments is not necessarily limited to the above-described systems. For example, in some embodiments, a microemulsion system, such as the reaction system used for microemulsion polymerization as described in U.S. Pat. No. 6,767,974, the entire disclosure of which is incorporated herein by reference, may be used as the reaction system.

Suitable catalysts for use in embodiments are not particularly limited, and include those that are known or discovered to be useful for selective hydrogenation of multiple bonds. For example, suitable catalysts for use in embodiments include palladium-based catalysts, such as Pd black, Pd/C, Pd/CaCO₃, Pd/Al₂O₃, ligated palladium catalysts and the like; Pt black; Pt/C; Raney Ni and the like, as well as mixtures thereof. The above-mentioned palladium-based catalysts are particularly suitable for some embodiments.

Alternatively, the catalyst of embodiments may be chosen from homogeneous catalysts. Suitable homogeneous catalysts include ruthenium complexes, such as RuCl₂(Ph₃P)₃; iridium complexes, such as HIrCl₂(Me₂SO)₃, Ir(CO)Br(Ph₃P)₂; rhodium complexes, such as RhCl(Ph₃As)₂; and platinum complexes, such as PtCl₂(Ph₃As)₂+SnCl₂H₂O.

The catalyst of embodiments may be one catalyst chosen from palladium-based catalysts, such as Pd black, Pd/C, Pd/CaCO₃, Pd/Al₂O₃ and the like; Pt black; Pt/C; Raney Ni; ruthenium complexes, such as RuCl₂(Ph₃P)₃; iridium complexes, such as HIrCl₂(Me₂SO)₃, Ir(CO)Br(Ph₃P)₂; rhodium complexes, such as RhCl(Ph₃As)₂; and platinum complexes, such as PtCl₂(Ph₃As)₂+SnCl₂H₂O; and the like. In embodiments, the catalyst may be a mixture of two or more such catalysts.

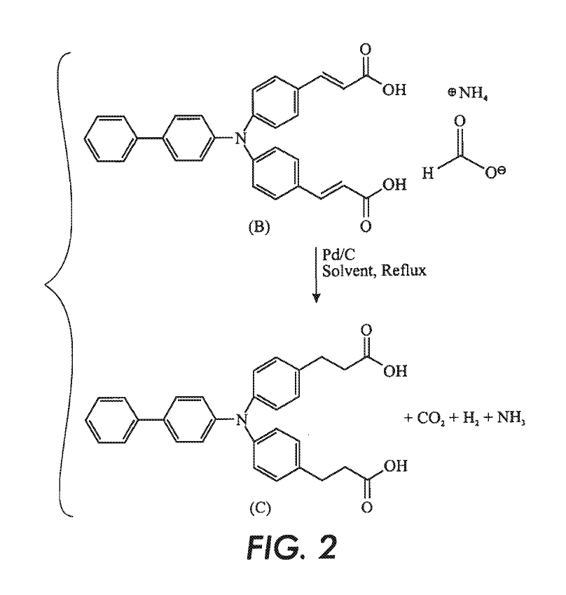

As an example, the reaction shown in FIG. 2 illustrates an embodiment in which an exemplary arylamine, N,N-di(propenoic acid)-4-aminobiphenyl (Compound B), is selectively hydrogenated by catalytic transfer hydrogenation using ammonium formate as a donor molecule and Pd/C as the catalyst. The reaction selectively reduces the double bonds in the acrylic acid portions of the N,N-di(propenoic acid)-4-aminobiphenyl (Compound B) to produce N,N-di(propanoic acid)-4-aminobiphenyl (Compound C).

However, the catalysts set forth above may be pyrophoric, such as for example, palladium on carbon (Pd/C), and may be dangerous when used in large scale reactions. Thus, in some embodiments, the catalyst may be chosen from catalytic systems such as, for example, metal hydride-transition metal salt catalyst systems.

It is well known that metal hydrides are useful for reducing functional groups in organic molecules. Used alone, metal hydrides show very little specificity in reducing functional groups; although individual metal hydrides may have distinct sets of functional groups that they are capable of reducing. For example, sodium borohydride, NaBH₄, will reduce only a very narrow range of organic functional groups, while lithium aluminum hydride, LiAlH₄, will reduce most organic functional groups. However, when metal hydrides are combined with transition metal salts, selective hydrogenation of carbon-carbon double bonds (C═C) in unsaturated ester molecules may be achieved. That is, selective catalysts that may be used in embodiments of processes disclosed herein include catalyst systems prepared from the reaction of a metal hydride and a transition metal salt, which may produce a black, granular material believed to be an active catalyst for selective hydrogenation of carbon-carbon double bonds (C═C) in α,β-unsaturated ester molecules.

The metal hydrides of metal hydride-transition metal salt catalyst systems of embodiments may have the general chemical formula MYH₄. In this formula, M may be chosen from alkali metals, such as, for example, elements of Group 1 (formerly Group IA) of the periodic table, including lithium (Li), sodium (Na), potassium (K), and the like; and organic functional groups, such as, for example, ammonium groups and the like. Y in the formula MYH₄ is an element chosen from Group 13 (formerly Group IIIA) of the periodic table, including boron (B), aluminum (Al), gallium (Ga), and the like. Examples of metal hydrides that may be used in the metal hydride-transition metal salt catalyst systems of embodiments include NaBH₄, LiBH₄, KBH₄, NH₄BH₄, (CH₃)₄NBH₄, NaAlH₄, LiAlH₄, KAlH₄, NaGaH₄, LiGaH₄, KGaH₄, and the like, and mixtures thereof. In some embodiments, the metal hydride is chosen from borohydrides and mixtures thereof; and in certain of these embodiments, the metal hydride is one or more borohydride chosen from sodium borohydride (NaBH₄), lithium borohydride (LiBH₄), potassium borohydride (KBH₄), ammonium borohydride (NH₄BH₄), tetramethyl ammonium borohydride ((CH₃)₄NBH₄), quaternary borohydrides, and mixtures thereof.

The transition metal salts of the metal hydride-transition metal salt catalyst systems of embodiments may be any suitable salts of transition metal elements, which are elements of Groups 3 through 12 (formerly Groups IIIB through IIB) of the periodic table. Representative examples of transition metals that may be useful in embodiments include elements in the scandium, titanium, vanadium, chromium, manganese, iron, cobalt, nickel, copper and zinc groups. In embodiments, the transition metal of the transition metal salts may be one or more of ruthenium, iron, cobalt, nickel, copper, manganese, rhodium, rhenium, platinum, palladium, chromium, silver, osmium, iridium, borides thereof, alloys thereof, and mixtures thereof. Non-limiting examples of suitable transition metal salts for use in embodiments include Cu₂Cl₂, CuCl₂, CoCl₂, PdCl₂, CuSO₄, and the like, and mixtures thereof.

In embodiments in which a metal hydride-transition metal salt catalyst system is employed, the metal hydride may be added slowly, to prevent H₂ build-up within the reaction system and to prevent the reaction from becoming exothermic. This may be accomplished, in some such embodiments, by dissolving the metal hydride in a solvent that contains a metal hydride stabilizing agent. Herein, the term “metal hydride stabilizing agent” refers to any component that retards, impedes, or prevents reaction of metal hydride with the solvent, which may be water in embodiments. Suitable metal hydride stabilizing agents may be hydroxide salts that are dissolved in a solvent prior to addition of the metal hydride. Examples of hydroxide salts for use in embodiments include sodium hydroxide (NaOH), lithium hydroxide (LiOH), potassium hydroxide (KOH), and the like and mixtures thereof. Examples of solvents for dissolving metal hydride stabilizing agents in embodiments include water; short-chain alcohols, such as methanol, ethanol, propanol, butanol and the like; polar aprotic solvents, such as dimethylformamide (DMF), dimethyl sulfoxide (DMSO), acetone, ethyl acetate, tetrahydrofuran (THF), methyl ethyl ketone (MEK) and the like; and mixtures thereof. In embodiments, for example, the solvent may be a mixture of methanol and tetrahydrofuran.

It should be understood that the metal hydride—transition metal salt catalyst systems described herein may be used individually, or as mixtures of multiple metal hydride—transition metal salt catalyst systems.

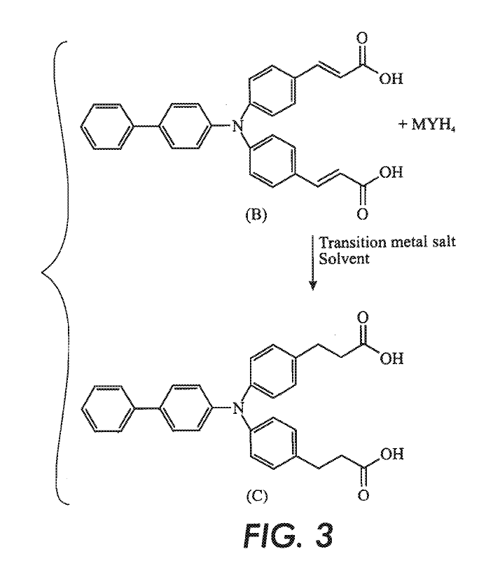

An exemplary, generalized reaction in which a metal hydride-transition metal salt catalyst system is used as the catalyst for selective hydrogenation of carbon-carbon double bonds (C═C) in an α,β-unsaturated ester molecule is shown in FIG. 3: The reaction selectively reduces the double bonds in the acrylic acid portions of the N,N-di(propenoic acid)-4-aminobiphenyl (Compound B) to produce N,N-di(propanoic acid)-4-aminobiphenyl (Compound C).

In embodiments, the catalyst may be included in amounts from about 2 to about 25% by weight, or from about 5 to about 15% by weight, based on the total weight of the reactants, i.e. based on the total weight of acceptor molecules, hydrogen donor molecules and catalyst.

The catalytic transfer hydrogenation reaction of embodiments may be carried out in any suitable organic solvent or mixture of organic solvents. Suitable organic solvents include, for example, alcohols, such as methanol, ethanol, isopropanol and the like; alkanes, such as hexane, decane and the like; ethers, such as diethyl ether, tetrahydrofuran, dimethoxyethane and the like; aromatic solvents, such as toluene, xylene, and the like; and mixtures thereof. The choice of specific solvent or mixture of solvents can be decided based on the solubility of the starting materials, intermediates and final products, and will be readily apparent or within routine experimentation to those skilled in the art. Solvents may be chosen based on the desired operating temperature range.

In embodiments, the solvent may be included in any suitable amount. In particular, solvent may be present in amounts from about 1 to about 99% by weight, or from about 25 to about 75% by weight, based on the total weight of the reactants. The amount of solvent can be readily determined by one of ordinary skill in the art, based on the amount of solvent necessary to dissolve the reactants.

Temperature may affect catalytic transfer hydrogenation in embodiments. For example, at higher temperatures (such as about 300° C. to about 350° C.), catalytic hydrogen transfer may be used to form aromatic groups. See, for example, Brieger, II.C. However, the reactions of embodiments may be carried out at temperatures of from about 0° C. to about 100° C., such as from about 20° C. to about 90° C. and from about 50° C. to about 65° C. The reaction temperature of embodiments may be selected to correspond to temperature at which the donor molecule may be oxidized, as described above.

The reaction of embodiments can be conducted in batch or continuous mode. However, in embodiments, the reaction is conducted in continuous mode. Continuous hydrogenation processes offer advantages in time, safety and cost over batch hydrogenation processes. For example, the reactor volume necessary for a continuous hydrogenation process may be significantly reduced, tip to 10 times, relative to that of a batch hydrogenation process, without sacrificing the reactor throughput. As discussed above, entire reactor systems for continuous hydrogenation can be located within a laboratory fumehood, taking relatively little space.

In the catalytic transfer hydrogenation reactions of embodiments, hydrogen gas is produced in-situ at low levels as a reaction by-product, as discussed above. Hydrogen gas production is proportional to the amount of hydrogen donor used, and a hydrogen atmosphere is not necessary for successful practice of embodiments. Because a hydrogen atmosphere is not required, the processes of embodiments can be practiced with greater production efficiency and safety than processes involving hydrogenation by compressed hydrogen gas. The processes of embodiments can achieve greater production efficiency, because standard reaction vessels may be used, without allowances for hydrogen gas volume in the reaction vessel. Processes of embodiments also increase safety because the low levels of hydrogen gas produced may be diluted with air or nitrogen and safely vented to the atmosphere, which can eliminate the need for special safety classifications within a production plant.

In addition, donor molecules suitable for use in embodiments generally present fewer safety risks. For example, one suitable donor molecule, ammonium formate, has a slightly higher health-hazard rating than hydrogen gas. However, hydrogen gas has a much greater flammability than ammonium formate, as illustrated by their respective National Fire Protection Association ratings, shown in Table 1.

TABLE 1

| | Health | Flammability | Reactivity |
|---|---|---|---|
| Hydrogen | 0 | 4 | 0 |
| Ammonium Formate | 1 | 0 | 0 |

After the reaction of embodiments is completed, suitable separation, filtration, and/or purification processes can be conducted, as desired to a desired purity level. For example, the desired arylamine product can be subjected to conventional organic washing steps, can be separated, can be decolorized (if necessary), treated with known absorbents (such as silica, alumina and clays, if necessary) and the like. The final product can be isolated, for example, by a suitable recrystallization procedure. The final product can also be dried, for example, by air drying, vacuum drying, or the like. All of these procedures are conventional and will be apparent to those skilled in the art.

The arylamines produced by these processes can be used as final products, or further processed and/or reacted to provide other compounds for their separate use. For example, the arylamine can be used itself as a charge-transport material in an electrostatographic imaging member, or it can be further processed and/or reacted to provide other charge-transport materials or other compounds useful in such electrostatographic imaging member. An exemplary electrostatographic imaging member will now be described in greater detail.

In electrophotographic photoreceptors of embodiments, the photoreceptors can include various layers such as undercoating layers, charge-generating layers, charge-transport layers, overcoat layers, and the like. The overcoating layers of embodiments can be a silicon-compound-containing overcoat layer, which can comprise one or more silicon compounds, a resin, and a charge-transport molecule such as an arylamine.

In embodiments, the resin may be a resin soluble in a liquid component in a coating solution used for formation of a silicon-compound-containing overcoat layer. Such a resin soluble in the liquid component may be selected based upon the kind of liquid component. For example, if the coating solution contains an alcoholic solvent, a polyvinyl acetal resin such as a polyvinyl butyral resin, a polyvinyl formal resin or a partially acetalized polyvinyl acetal resin in which butyral is partially modified with formal or acetoacetal, a polyamide resin, a cellulose resin such as ethyl cellulose and a phenol resin may be suitably chosen as the alcohol-soluble resins. These resins may be used either alone or as a combination of two or more resins. Of the above-mentioned resins, the polyvinyl acetal resin is particularly suitable in embodiments in terms of electric characteristics.

In embodiments, the weight-average molecular weight of the resin soluble in the liquid component may be from about 2,000 to about 1,000,000, such as from about 5,000 to about 50,000. When the weight-average molecular weight is less than about 2,000, enhancing discharge-gas resistance, mechanical strength, scratch resistance, particle dispersibility, etc., tend to become insufficient. However, when the weight-average molecular weight exceeds about 1,000,000, the resin solubility in the coating solution decreases, and the amount of resin added to the coating solution may be limited and poor film formation in the production of the photosensitive layer may result.

Further, the amount of the resin soluble in the liquid component may be, in embodiments, from about 0.1 to about 15% by weight, or from about 0.5 to about 10% by weight, based on the total amount of the coating solution. When the amount added is less than 0.1% by weight, enhancing discharge-gas resistance, mechanical strength, scratch resistance, particle dispersibility, etc., tend to become insufficient. However, if the amount of the resin soluble in the liquid component exceeds about 15% by weight, there is a tendency for formation of indistinct images when the electrophotographic photoreceptor of the disclosure is used in high-temperature and high-humidity environments.

There is no particular limitation on the silicon compound used in silicon-compound-containing layers of disclosed embodiments, as long as it has at least one silicon atom. However, a compound having two or more silicon atoms in its molecule may be used in embodiments. The use of compounds having two or more silicon atoms in its molecule allows both the strength and image quality of the electrophotographic photoreceptor to be achieved at higher levels.

Further, in embodiments, the silicon compounds may include silane coupling agents such as a tetrafunctional alkoxysilane, such as tetramethoxysilane, tetraethoxysilane and the like; a trifunctional alkoxysilane such as methyltrimethoxysilane, methyltriethoxysilane, ethyltrimethoxysilane, methyltrimethoxyethoxysilane, vinyltrimethoxysilane, vinyltriethoxysilane, phenyltrimethoxysilane, γ-glycidoxypropylmethyldiethoxysilane, γ-glycidoxypropyltrimethoxysilane, γ-glycidoxypropyltriethoxysilane, γ-aminopropyltriethoxysilane, γ-aminopropyltrimethoxysilane, γ-aminopropylmethyldimethoxysilane, N-β-(aminoethyl)-γ-aminopropyltriethoxysilane, (tridecafluoro-1,1,2,2-tetrahydrooctyl)triethoxysilane, (3,3,3-trifluoropropyl)trimethoxysilane, 3-(heptafluoroisopropoxy)propyltriethoxysilane, 1H,1H,2H,2H-perfluoroalkyltriethoxysilane, 1H,1H,2H,2H-perfluorodecyltriethoxysilane or 1H,1H,2H,2H-perfluorooctyltriethoxysilane; a bifunctional alkoxysilane such as dimethyldimethoxysilane, diphenyldimethoxysilane or methylphenyldimethoxysilane; and a monofunctional alkoxysilane such as trimethylmethoxysilane. In order to improve the strength of the photosensitive layer, trifunctional alkoxysilanes and tetrafunctional alkoxysilanes may be used in embodiments, and in order to improve the flexibility and film-forming properties, monofunctional alkoxysilanes and bifunctional alkoxysilanes may be used in embodiments.

Silicone hard-coating agents containing these coupling agents can also be used in embodiments. Commercially available hard-coating agents include KP-85, X-40-9740 and X-40-2239 (available from Shinetsu Silicone Co., Ltd.), and AY42-440, AY42-441 and AY49-208 (available from Toray Dow Corning Co., Ltd.).

Various fine particles can also be added to the silicon-compound-containing layer, for example, to further improve the stain adhesion resistance and lubricity of embodiments of the electrophotographic photoreceptor. The fine particles may be used either alone or as a combination of two or more such fine particles. Non-limiting examples of the fine particles include fine particles containing silicon, such as fine particles containing silicon as a constituent element, and specifically include colloidal silica and fine silicone particles. The content of the fine silicone particles in the silicon-compound-containing layer of embodiments may be within the range of 0.1 to 20% by weight, or within the range of 0.5 to 10% by weight, based on the total solid content of the silicon-compound-containing layer.

Colloidal silica used in embodiments as the fine particles containing silicon in the disclosure is selected from an acidic or alkaline aqueous dispersion of the fine particles having an average particle size of 1 to 100 nm, or 10 to 30 nm, and a dispersion of the fine particles in an organic solvent, such as an alcohol, a ketone or an ester, and, generally, commercially available particles can be used.

There is no particular limitation on the solid content of colloidal silica in a top-surface layer of the electrophotographic photoreceptor of embodiments. However, in embodiments, colloidal silica may be included in amounts of from about 1 to about 50% by weight, such as from about 5 to about 30% by weight, based on the total solid content of the top surface layer, in terms of film forming properties, electric characteristics and strength.

The fine silicone particles used as the fine particles containing silicon in the disclosure may be selected from silicone resin particles, silicone rubber particles and silica particles surface-treated with silicone, which are spherical and have an average particle size of from about 1 to 500 nm, such as from about 10 to about 100 mm, and generally, commercially available particles can be used in embodiments.

In embodiments, the fine silicone particles are small-sized particles that are chemically inactive and excellent in dispersibility in a resin, and further are low in content as may be necessary for obtaining sufficient characteristics. Accordingly, the surface properties of the electrophotographic photoreceptor can be improved without inhibition of the cross-linking reaction. That is to say, fine silicone particles improve the lubricity and water repellency of electrophotographic photoreceptor surfaces where incorporated into strong cross-linked strictures, which may then be able to maintain good wear resistance and stain-adhesion resistance for a long period of time. The content of the fine silicone particles in the silicon-compound-containing layer of embodiments may be from about 0.1 to about 20% by weight, such as from about 0.5 to about 10% by weight, based on the total solid content of the silicon-compound-containing layer.

Other fine particles that may be used in embodiments include fine fluorine-based particles such as ethylene tetrafluoride, ethylene trifluoride, propylene hexafluoride, vinyl fluoride and vinylidene fluoride, and semiconductive metal oxides such as ZnO—Al₂O₃, SnO₂—Sb₂O₃, In₂O₃—SnO₂, ZnO—TiO₂, MgO—Al₂O₃, FeO—TiO₂, TiO₂, SnO₂, In₂O₃, ZnO and MgO.

In conventional electrophotographic photoreceptors, when the above-mentioned fine particles are contained in the photosensitive layer, the compatibility of the fine particles with a charge-transport substance or a binding resin may become insufficient, which causes layer separation in the photosensitive layer, and thus the formation of an opaque film. As a result, the electric characteristics have deteriorated in some cases. In contrast, the silicon-compound-containing layer of embodiments (a charge-transport layer in this case) may contain the resin soluble in the liquid component in the coating solution used for formation of this layer and the silicon compound, thereby improving the dispersibility of the fine particles in the silicon-compound-containing layer. Accordingly, the pot life of the coating solution may be sufficiently prolonged, and deterioration of the electric characteristics may be prevented.

Further, an additive such as a plasticizer, a surface modifier, an antioxidant, or an agent for preventing deterioration by light can also be used in the silicon-compound-containing layer of embodiments. Non-limiting examples of plasticizers that may be used in embodiments include, for example, biphenyl, biphenyl chloride, terphenyl, dibutyl phthalate, diethylene glycol phthalate, dioctyl phthalate, triphenylphosphoric acid, methylnaphthalene, benzophenone, chlorinated paraffin, polypropylene, polystyrene and various fluorohydrocarbons.

The antioxidants may include an antioxidant having a hindered-phenol, hindered-amine, thioether or phosphite partial structure. This is effective for improvement of potential stability and image quality in environmental variation. The antioxidants include an antioxidant having a hindered-phenol, hindered-amine, thioether or phosphite partial structure. This is effective for improvement of potential stability and image quality in environmental variation. For example, the hindered-phenol antioxidants include SUMILIZER BHT-R, SUMILIZER MDP-S, SUMILIZER BBM-S, SUMILIZER WX-R, SUMILIZER NW, SUMILIZER BP-76, SUMILIZER BP-101, SUMILIZER GA-80. SUMILIZER GM and SUMILIZER GS (the above are manufactured by Sumitomo Chemical Co., Ltd.), IRGANOX 1010, IRGANOX 1035, IRGANOX 1076, IRGANOX 1098, IRGANOX 1135, IRGANOX 1141, IRGANOX 1222, IRGANOX 1330, IRGANOX 1425WLj, IRGANOX 1520Lj, IRGANOX 245, IRGANOX 259, IRGANOX 3114, IRGANOX 3790, IRGANOX 5057 and IRGANOX 565 (the above are manufactured by Ciba Specialty Chemicals), and ADECASTAB AO-20, ADECASTAB AO-30, ADECASTAB AO-40, ADECASTAB AO-50, ADECASTAB AO-60, ADECASTAB AO-70, ADECASTAB AO-80 and ADECASTAB AO-330i (the above are manufactured by Asahi Denka Co., Ltd.). The hindered-amine antioxidants include SANOL LS2626, SANOL LS765, SANOL LS770, SANOL LS744, TINUVIN 144, TINUVIN 622LD, MARK LA57, MARK LA67, MARK LA62, MARK LA68, MARK LA63 and SUMILIZER TPS, and the phosphite antioxidants include MARK 2112, MARK PEP 8, MARK PEP 24G, MARK PEP 36, MARK 329K and MARK HP 10. Of these, hindered-phenol and hindered-amine antioxidants may be particularly suitable, in embodiments.

There is no particular limitation on the thickness of the silicon-compound-containing layer, however, in embodiments, the silicon-compound-containing layer may be from about 2 to about 5 μm in thickness, such as from about 2.7 to about 3.2 μm in thickness.

The electrophotographic photoreceptor of embodiments may be either a function-separation-type photoreceptor, in which a layer containing a charge-generating substance (charge-generating layer) and a layer containing a charge-transport substance (charge-transport layer) are separately provided, or a monolayer-type photoreceptor, in which both the charge-generating layer and the charge-transport layer are contained in the same layer, as long as the electrophotographic photoreceptor of the particular embodiment has the photosensitive layer provided with the above-mentioned silicon-compound-containing layer. The electrophotographic photoreceptor will be described in greater detail below, taking the function-separation-type photoreceptor as an example.

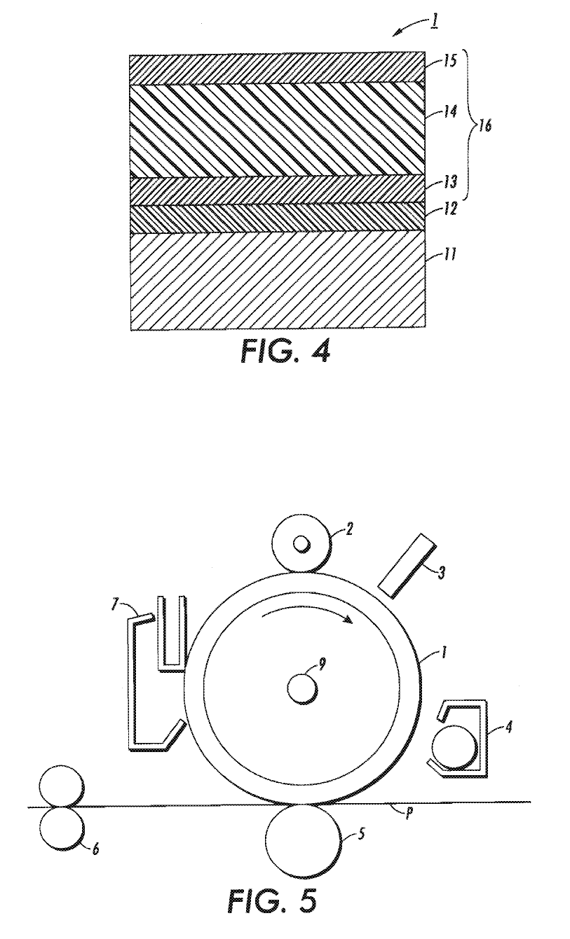

FIG. 4 is a cross-sectional view schematically showing an embodiment of the electrophotographic photoreceptor of the disclosure. The electrophotographic photoreceptor 1 shown in FIG. 4 is a function-separation-type photoreceptor in which a charge-generating layer 13 and a charge-transport layer 14 are separately provided. That is, the charge-generating layer 13, the charge transport layer 14 and a protective layer 15 from a photosensitive layer 16 laminated onto an underlayer 12 on a conductive support 11. The protective layer 15 contains a resin soluble in the liquid component contained in the coating solution used for formation of this layer and the silicon compound. The various layers of the photoreceptor shown in FIG. 4 are generally known, and are described in detail in the above-mentioned commonly owned and co-pending applications.

The electrophotographic photoreceptor of embodiments should not be construed as being limited to the above-mentioned constitution. For example, the electrophotographic photoreceptor shown in FIG. 4 is provided with the protective layer 15. However, when the charge-transport layer 14 contains the resin soluble in the liquid component in the coating, solution used for formation of this layer and the silicon compound, the charge-transport layer 14 may be used as a top surface layer (a layer on the side farthest apart from the support 11) without using the protective layer 15. In this case, the charge-transport substance contained in the charge-transport layer 14 is desirably soluble in the liquid component in the coating solution used for formation of the charge-transport layer 14. For example, when the coating solution used for formation of the charge-transport layer 14 contains an alcohol solvent, the silicon compounds described above, including arylamine derivatives prepared by processes that include selective hydrogenation by catalytic transfer, can be used as the charge-transport substances. In embodiments, a particularly suitable charge-transport molecule is the following arylamine (Compound D), which may be produced from Compound C:

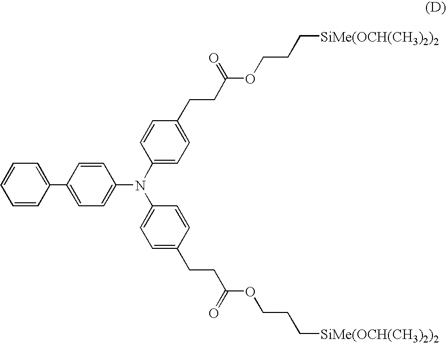

FIG. 5 is a schematic view showing an embodiment of an image forming apparatus. In the apparatus shown in FIG. 5, the electrophotographic photoreceptor 1 constituted as shown in FIG. 4 is supported by a support 9, and rotatable at a specified rotational speed in the direction indicated by the arrow, centered on the support 9. A contact-charging device 2, an exposure device 3, a developing device 4, a transfer device 5 and a cleaning unit 7 are arranged in this order along the rotational direction of the electrophotographic photoreceptor 1. Further, this exemplary apparatus is equipped with an image-fixing device 6, and a medium P to which a toner image is to be transferred is conveyed to the image-fixing device 6 through the transfer device 5.

The contact-charging device 2 has a roller-shaped contact-charging member. The contact-charging member is arranged so that it comes into contact with a surface of the photoreceptor 1, and a voltage is applied, thereby being able to give a specified potential to the surface of the photoreceptor 1. In embodiments, a contact-charging member may be formed from a metal such as aluminum, iron or copper, a conductive polymer material such as a polyacetylene, a polypyrrole or a polythiophene, or a dispersion of fine particles of carbon black, copper iodide, silver iodide, zinc sulfide, silicon carbide, a metal oxide or the like in an elastomer material such as polyurethane rubber, silicone rubber, epichlorohydrin rubber, ethylene-propylene rubber, acrylic rubber, fluororubber, styrene-butadiene rubber or butadiene rubber. Non-limiting examples of metal oxides that may be used in embodiments include ZnO, SnO₂, TiO₂, In₂O₃, MoO₃ and complex oxides thereof. Further, a perchlorate may be added to the elastomer material to impart conductivity.

Further, a covering layer can also be provided on a surface of the contact-charging member of embodiments. Non-limiting examples of materials that may be used in embodiments for forming a covering layer include N-alkoxy-methylated nylon, cellulose resins, vinylpyridine resins, phenol resins, polyurethanes, polyvinyl butyrals, melamines and mixtures thereof. Furthermore, emulsion resin materials such as acrylic resin emulsions, polyester resin emulsions or polyurethanes, may be used. In order to further adjust resistivity, conductive agent particles may be dispersed in these resins, and in order to prevent deterioration, an antioxidant can also be added thereto. Further, in order to improve film-forming properties in forming the covering layer, a leveling agent or a surfactant may be added to the emulsion resin in embodiments.

The resistance of the contact-charging member of embodiments may be from 10⁰ to 10¹⁴ Ωcm, and from 10² to 10¹² Ωcm. When a voltage is applied to this contact-charging member, either a DC voltage or an AC voltage can be used as the applied voltage. Further, a superimposed voltage of a DC voltage and an AC voltage can also be used.

In the exemplary apparatus shown in FIG. 5, the contact-charging member of the contact-charging device 2 is in the shape of a roller. However, such a contact-charging member may be in the shape of a blade, a belt, a brush or the like.

Further, in embodiments an optical device that can perform desired imagewise exposure to a surface of the electrophotographic photoreceptor 1 with a light source such as a semiconductor laser, an LED (light emitting diode) or a liquid crystal shutter, may be used as the exposure device 3.

Furthermore, a known developing device using a normal or reversal developing agent of a one-component system, a two-component system or the like may be used in embodiments as the developing device 4. There is no particular limitation on toners that may be used in embodiments.

Contact-type transfer-charging devices using a belt, a roller, a film, a rubber blade or the like, or a scorotron-transfer charger or a corotron-transfer charger utilizing corona discharge may be employed as the transfer device 5, in various embodiments.

Further, in embodiments, the cleaning device 7 may be a device for removing a remaining toner adhered to the surface of the electrophotographic photoreceptor 1 after a transfer step, and the electrophotographic photoreceptor 1 repeatedly subjected to the above-mentioned image formation process may be cleaned thereby. In embodiments, the cleaning device 7 may be a cleaning blade, a cleaning brush, a cleaning roll or the like. Materials for the cleaning blade include urethane rubber, neoprene rubber and silicone rubber.

In the exemplary image forming device shown in FIG. 5, the respective steps of charging, exposure, development, transfer and cleaning are conducted in turn in the rotation step of the electrophotographic photoreceptor 1, thereby repeatedly performing image formation. The electrophotographic photoreceptor 1 may be provided with specified silicon-compound-containing layers and photosensitive layers that have excellent discharge gas resistance, mechanical strength, scratch resistance, particle dispersibility, etc. Accordingly, even in embodiments in which the photoreceptor is used together with the contact-charging device or the cleaning blade, or further with spherical toner obtained by chemical polymerization, good image quality can be obtained without the occurrence of image defects such as fogging. That is, embodiments provide image forming apparatuses that can stably provide good image quality for a long period of time is realized.

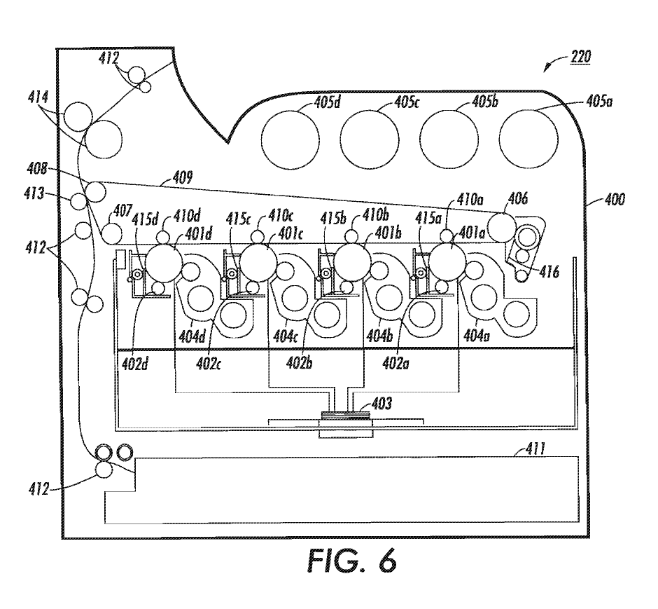

FIG. 6 is a cross-sectional view showing another exemplary embodiment of an image-forming apparatus. The image-forming apparatus 220 shown in FIG. 6 is an image-forming apparatus of an intermediate-transfer system, and four electrophotographic photoreceptors 401a to 401d are arranged in parallel with each other along an intermediate-transfer belt 409 in a housing 400.

Here, the electrophotographic photoreceptors 401a to 401d carried by the image-forming apparatus 220 are each the electrophotographic photoreceptors. Each of the electrophotographic photoreceptors 401a to 401d may rotate in a predetermined direction (counterclockwise on FIG. 6), and charging rolls 402a to 402d, developing device 404a to 404d, primary transfer rolls 410a to 410d and cleaning blades 415a to 415d are each arranged along the rotational direction thereof. In each of the developing device 404a to 404d, four-color toners of yellow (Y), magenta (M), cyan (C) and black (B) contained in toner cartridges 405a to 405d can be supplied, and the primary transfer rolls 410a to 410d are each brought into abutting contact with the electrophotographic photoreceptors 401a to 401d through an intermediate-transfer belt 409.

Further, a laser-light source (exposure unit) 403 is arranged at a specified position in the housing 400, and it is possible to irradiate surfaces of the electrophotographic photoreceptors 401a to 401d after charging with laser light emitted from the laser-light source 403. This performs the respective steps of charging, exposure, development, primary transfer and cleaning in turn in the rotation step of the electrophotographic photoreceptors 401a to 401d, and toner images of the respective colors are transferred onto the intermediate-transfer belt 409, one over the other.

The intermediate-transfer belt 409 is supported with a driving roll 406, a backup roll 408 and a tension roll 407 at a specified tension, and rotatable by the rotation of these rolls without the occurrence of deflection. Further, a secondary transfer roll 413 is arranged so that it is brought into abutting contact with the backup roll 408 through the intermediate-transfer belt 409. The intermediate-transfer belt 409, which has passed between the backup roll 408 and the secondary transfer roll 413, is cleaned up by a cleaning blade 416, and then repeatedly subjected to the subsequent image-formation process.

Further, a tray (tray for a medium to which a toner image is to be transferred) 411 is provided at a specified position in the housing 400. The medium to which the toner image is to be transferred (such as paper) in the tray 411 is conveyed in turn between the intermediate-transfer belt 409 and the secondary transfer roll 413, and further between two fixing rolls 414 brought into abutting contact with each other, with a conveying roll 412, and then delivered out of the housing 400.

According to the exemplary image-forming apparatus 220 shown in FIG. 6, the use of electrophotographic photoreceptors of embodiments as electrophotographic photoreceptors 401a to 401d may achieve discharge gas resistance, mechanical strength, scratch resistance, etc. on a sufficiently high level in the image-formation process of each of the electrophotographic photoreceptors 401a to 401d. Accordingly, even when the photoreceptors are used together with the contact-charging devices or the cleaning blades, or further with the spherical toner obtained by chemical polymerization, good image quality can be obtained without the occurrence of image defects such as fogging. Therefore, also according to the image-forming apparatus for color-image formation using the intermediate-transfer body, such as this embodiment, the image-forming apparatus, which can stably provide good image quality for a long period of time, is realized.

The disclosure should not be construed as being limited to the above-mentioned embodiments. For example, each apparatus shown in FIG. 5 or 6 may be equipped with a process cartridge comprising the electrophotographic photoreceptor 1 (or the electrophotographic photoreceptors 401a to 401d) and charging device 2 (or the charging devices 402a to 402d). The use of such a process cartridge allows maintenance to be performed more simply and easily.

Further, in embodiments, when a charging device of the non-contact charging system such as a corotron charger is used in place of the contact-charging device 2 (or the contact-charging devices 402a to 402d), sufficiently good image quality can be obtained.

Specific examples are described in detail below. These examples are intended to be illustrative, and the materials, conditions, and process parameters set forth in these exemplary embodiments are not limiting. All parts and percentages are by weight unless otherwise indicated.

### EXAMPLES

### Example 1

### Batch Pd/C-Catalyzed Hydrogenation of Trans-Cinnamic Acid

Trans-cinnamic acid, which is analogous to the di(propenoic acid portions of N,N-di(propenoic acid)-4-aminobiphenyl (Compound B), is hydrogenated as a model system. In a 500-mL glass beaker, 29.6 grams of trans-cinnamic acid (Aldrich; 99+%), was dissolved in a mixture of 150 grains of methanol and 150 grams of tetrahydrofuran by stirring. To this solution, 0.2 grams of 10% palladium on charcoal (Pd/C; Aldrich; 10 wt % on a dry basis) was added to make a slurry. This slurry was charged into a 1-L stainless steel Buchi reactor. Mixing was started at 400 rpm using a pitch blade impeller. The reaction jacket temperature was maintained at 25° C., and the reactor was pressurized to 500 kPa using hydrogen gas. Reaction progress was measured by pressure drop in the reactor as hydrogen was consumed. Hydrogen was added at intervals, and the reaction was considered complete when the pressure stopped dropping. The slurry was discharged from the reactor, and filtered through a 0.5 μm fluorinated filter membrane. UV and ¹H NMR analysis showed that hydrocinnamic acid was produced.

### Example 2

### Continuous Pd/C-Catalyzed Hydrogenation of Trans-Cinnamic Acid

A continuous tubular reactor system as described above with respect to FIG. 1 was prepared by filling the packed bed portion of the reactor with a mixture of approximately 1:5 catalyst:sand, by dry mixing 5 ml (˜2.5 grams) of 10% palladium on charcoal (Pd/C; Aldrich; 10 wt % on a dry basis) with 25 ml white quartz sand (50+70 mesh; Aldrich) in a beaker with a spatula. This mixture was poured into the two linear portions of the tubular reactor, with inline filters acting as supports to hold the mixture in the tubular reactor. The curved portions of the reactor were not filled with the catalyst:sand mixture. Reactor assembly was then completed. Once the reactor assembly was complete, the reactor was filled with a 50:50 (by weight) mixture of methanol and tetrahydrofuran to achieve flow through the reaction system.

In a 500-mL glass beaker, 29.6 grams of trans-cinnamic acid (Aldrich; 99+%), was dissolved in a mixture of 150 grams of methanol and 150 grams of tetrahydrofuran by stirring. The beaker containing the solution was placed on a weigh scale; the reactor inlet line was placed in the solution; and the beaker was covered to minimize evaporation. The reactor system was pressurized to 500 kPa using hydrogen gas via a hydrogen regulator. The liquid pump was started to feed the solution through the reactor at the desired rate. Reaction progress was measured by mass loss on the weigh scale. Samples were removed from the reactor at intervals. UV and ¹H NMR analysis showed that hydrocinnamic acid was produced. The reactor was shut down by turning off the liquid pump, closing the hydrogen regulator, and bleeding hydrogen from the system.

### Example 3

### Batch Pd/C-Catalyzed Hydrogenation of N,N-di(propenoic acid)-4-aminobiphenyl (Compound B)

Into a 500-mL round-bottom flask, the following is charged: 10.0 grams (21.667 mmol) of N,N-di(propenoic acid)-4-aminobiphenyl (Compound B), 10.25 grams of ammonium formate, 2.5 grams of 10% palladium on charcoal (Pd/C), 200 mL of methanol and 50 mL of tetrahydrofuran. The mixture is heated to reflux in a water bath. The reaction may be monitored by thin layer chromatography. The reaction solution is cooled and filtered. The solvent is removed under nitrogen. The solid residue is washed with water and dried. The ethyl acetate is removed under nitrogen to recover the product, N,N-di(propanoic acid)-4-aminobiphenyl (Compound C).

### Example 4

### Continuous Pd/C-Catalyzed Hydrogenation of N,N-di(propenoic acid)-4-aminobiphenyl (Compound B)

Into a continuous tubular reactor having a fixed catalyst bed of 10% palladium on charcoal (Pd/C) as described above in Example 1, a solution of N,N-di(propenoic acid)-4-aminobiphenyl (Compound B) in a 50:50 by weight mixture of methanol and tetrahydrofuran is charged. The reactor is pressurized to 500 kPa using hydrogen gas via a hydrogen regulator. The liquid pump is started to feed the solution through the reactor at the desired rate. Reaction progress is measured by mass loss on the weigh scale.

Comparison and Analysis of Examples 1-4

Based on the results of Examples 1-4, the continuous hydrogenation reactions of this disclosure are able to meet or exceed the throughput of large scale batch reactions. For example, a 1-L reactor can produce approximately 45 g of Compound C each day, and a 10-L reactor can produce approximately 450 g of Compound C each day. As shown below in Table 2 (which contains calculations relating to scaling the process shown in Example 3), continuous reaction processes allow a more efficient synthesis of, for example, Compound C.

TABLE 2

| | | | | Calculated Continuous Process Throughput | | | |
|---|---|---|---|---|---|---|---|
| Continuous Tubular | | | | Volumetric | Volumetric | Volumetric | Rate of |
| Reactor Sizing | | | | flow rate | flow rate | flow rate | throughput |
| Internal | Internal | | Reactor | of reaction | of reaction | of reaction | (100% |
| diameter | diameter | Length | volume | mixture | mixture | mixture | conversion) |
| (inches) | (cm) | (cm) | (cc) | (cc/min) | (cc/hour) | (cc/day) | (cc/day) |
| 0.25 | 0.635 | 30.48 | 9.65 | 0.16 | 9.6 | 48 | 3.9 |
| 0.50 | 1.27 | 30.48 | 38.59 | 0.64 | 38.6 | 193 | 15.4 |
| 1.00 | 2.54 | 30.48 | 154.37 | 2.57 | 154.4 | 772 | 61.7 |
| 1.50 | 3.81 | 30.48 | 347.32 | 5.79 | 347.3 | 1737 | 138.9 |
| 2.00 | 5.08 | 30.48 | 617.46 | 10.29 | 617.5 | 3087 | 247.0 |
| 2.50 | 6.35 | 30.48 | 964.79 | 16.08 | 964.8 | 4824 | 385.9 |
| 3.00 | 7.62 | 30.48 | 1389.30 | 23.15 | 1389.3 | 6946 | 555.7 |

### Comparative Example

### Hydrogenation of N,N-di(propenoic acid)-4-aminobiphenyl (Compound B)

Into a 10-L glass reactor, the following was charged: 1385.0 grams (2.83 mol) of N,N-di(propenoic acid)-4-aminobiphenyl (Compound B), 3 L of toluene, 2 L of methanol and 0.5 L of tetrahydrofuran, and 6 grams of 10% palladium on charcoal (Pd/C) were added. The mixture was placed under a nitrogen atmosphere and stirred vigorously for five (5) days. The end of the reaction was determined by NMR. The reaction solution was cooled and filtered to remove the catalyst. The solvent was removed under reduced pressure. 1330 grams of crude product was obtained. The product was recrystallized from 1 L of acetone, 1 L of butanol and 1.5 L of methanol to obtain 908 g (1.84 mol) of an uncolored solid. ¹H NMR analysis showed that N,N-di(propanoic acid)-4-aminobiphenyl (Compound C) was produced.

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A process for selectively hydrogenating double and/or triple bonds in organic molecules, comprising:
providing one or more acceptor molecules that contains one or more alkyl groups having one or more double bonds and/or one or more triple bonds;
providing one or more hydrogen donor molecules; and
supplying a mixed feed of said acceptor molecules and said hydrogen donor molecules to a reactor including a fixed catalyst bed and thereby hydrogenating said double bonds and/or said triple bonds of said organic molecules in the presence of one or more catalysts.

2. The process according to claim 1, wherein process is performed continuously.

3. The process according to claim 1, wherein said process is performed as a batch process.

4. The process according to claim 1, wherein said acceptor molecule is a substituted triarylamine compound.

5. The process according to claim 2, wherein said substituted triarylamine compound is an ester-derivatized triarylamine compound.

6. The process according to claim 2, wherein said substituted triarylamine compound is a disubstituted 4-aminobiphenyl compound.

7. The process according to claim 2, wherein said substituted triarylamine compound is a N,N-di(alkylacrylic acid)-4-aminobiphenyl compound.

8. The process according to claim 1, wherein said hydrogen donor molecule is present in an amount of from about 1 to about 10 molar equivalents, based on an amount of acceptor molecules.

9. The process according to claim 8, wherein said hydrogen donor molecule is present in an amount of from about 1 to about 4 molar equivalents, based on an amount of acceptor molecules.

10. The process according to claim 1, wherein said hydrogen donor molecule is one or more donor molecule selected from the group consisting of hydrazine, formic acid, formates, substituted and unsubstituted cyclohexenes, substituted and unsubstituted octalins, substituted and unsubstituted tetralins, substituted and unsubstituted pinenes, substituted and unsubstituted careens, substituted and unsubstituted phellandrenes, substituted and unsubstituted terpinolenes, substituted and unsubstituted menthenes, substituted and unsubstituted cadalene, substituted and unsubstituted pulegones, substituted and unsubstituted selinenes, alcohols, and mixtures thereof.

11. The process according to claim 10, wherein said hydrogen donor molecule is ammonium formate.

12. The process according to claim 1, wherein said catalyst is one or more catalyst selected from the group consisting of palladium-based catalysts; Pt black, Pt/C, Raney Ni, RuCl₂(Ph₃P)₃, HIrCl₂(Me₂SO)₃, Ir(CO)Br(Ph₃P)₂, RhCl(Ph₃As)₂, PtCl₂(Ph₃As)₂+SnCl₂H₂O, and mixtures thereof.

13. The process according to claim 12, wherein said catalyst is one or more catalyst selected from the group consisting of palladium-based catalysts and mixtures thereof.

14. The process according to claim 12, wherein said catalyst is one or more catalyst selected from the group consisting of Pd black, Pd/C, Pd/CaCO₃, Pd/Al₂O₃, ligated palladium catalysts, Pt black, Pt/C, Raney Ni, and mixtures thereof.

15. The process according to claim 1, wherein said catalyst is one or more catalyst selected from the group consisting of metal hydride-transition metal salt catalyst systems, and mixtures thereof.

16. The process according to claim 15, wherein said metal hydride-transition metal salt catalyst systems comprises a metal hydride selected from the group consisting of NaBH₄, LiBH₄, KBH₄, NH₄BH₄, (CH₃)₄NBH₄, NaAlH₄, LiAlH₄, KAlH₄, NaGaH₄, LiGaH₄, KGaH₄, quaternary borohydrides, ion exchange resins and mixtures thereof.

17. The process according to claim 15, wherein said metal hydride-transition metal salt catalyst systems comprises a transition metal salt that includes a transition metal selected from the group consisting of ruthenium, iron, cobalt, nickel, copper, manganese, rhodium, rhenium, platinum, palladium, chromium, silver, osmium, iridium, borides thereof alloys thereof, and mixtures thereof.

18. The process according to claim 17, wherein said transition metal salt is selected from the group consisting of Cu₂Cl₂, CuCl₂, CoCl₂, PdCl₂, CuSO₄, and mixtures thereof.

19. The process according to claim 1, wherein said catalyst is present in an amount of from 2 to about 25% by weight, based on the total weight of acceptor molecules, hydrogen donor molecules and catalyst.

20. The process according to claim 1, wherein said hydrogenating is carried out at a temperature of from about 0° C. to about 100° C.

21. The process according to claim 20, wherein said hydrogenating is carried out at a temperature of from about 20° C. to about 90° C.

22. The process according to claim 20, wherein said hydrogenating is carried out at a temperature of from about 50° C. to about 65° C.

23. The process according to claim 1, wherein said hydrogenating is carried out in one or more organic solvent selected from the group consisting of alcohols, alkanes, ethers, aromatic solvents, and mixtures thereof.

24. The process according to claim 23, wherein said hydrogenating is carried out in one or more organic solvent selected from the group consisting of methanol, ethanol, isopropanol, hexane, decane, diethyl ether, tetrahydrofuran, dimethoxyethane, toluene, xylene, and mixtures thereof.

25. A process for preparing arylamine molecules, comprising:
selectively hydrogenating bonds in one or more arylamine compounds, wherein selectively hydrogenating bonds comprises:
providing one or more arylamine compounds that contains one or more alkyl groups having one or more double bonds and/or one or more triple bonds;
providing one or more hydrogen donor molecules; and
supplying a mixed feed of said acceptor molecules and said hydrogen donor molecules to a reactor including a fixed catalyst bed and thereby hydrogenating said double bonds and/or said triple bonds of said organic molecules in the presence of one or more catalysts.

26. The process according to claim 25, wherein said selectively hydrogenating is performed continuously.

27. The process according to claim 25, wherein said selectively hydrogenating is performed as a batch process.

28. The process according to claim 25, wherein said arylamine compounds are one or more disubstituted triarylamine compounds.

29. The process according to claim 25, wherein said disubstituted triarylamine compounds are one or more N,N-di(alkylacrylic acid)-4-aminobiphenyl compounds.

30. The process according to claim 25, wherein said hydrogenating is carried out at a temperature of from about 0° C. to about 100° C.

31. The process according to claim 25, wherein said catalysts are one or more catalysts selected from the group consisting of palladium-based catalysts.

32. The process according to claim 25, wherein said catalyst is one or more catalyst selected from the group consisting of metal hydride-transition metal salt catalyst systems, and mixtures thereof.

33. The process according to claim 25, wherein said hydrogen donor molecules are one or more donor molecules selected from the group consisting of hydrazine, formic acid, formates, substituted and unsubstituted cyclohexenes, substituted and unsubstituted octalins, substituted and unsubstituted tetralins, substituted and unsubstituted pinenes, substituted and unsubstituted careens, substituted and unsubstituted phellandrenes, substituted and unsubstituted terpinolenes, substituted and unsubstituted menthenes, substituted and unsubstituted cadalene, substituted and unsubstituted pulegones, substituted and unsubstituted selinenes, alcohols, and mixtures thereof.

34. The process according to claim 25, wherein said hydrogenating is carried out in one or more organic solvents selected from the group consisting of alcohols, alkanes, ethers, aromatic solvents, and mixtures thereof.

35. An electrophotographic imaging member comprising:
a substrate;
a charge-generating layer;
a charge-transport layer; and
optionally an overcoating layer; wherein:
said charge-transporting layer includes one or more charge-transport molecules prepared by a process that comprises:
providing one or more precursor molecules that contains one or more alkyl groups having one or more double bonds and/or one or more triple bonds;
providing one or more hydrogen donor molecules; and
supplying, a mixed feed of said acceptor molecules and said hydrogen donor molecules to a reactor including a fixed catalyst bed and thereby hydrogenating said double bonds and/or said triple bonds of said organic molecules in the presence of one or more catalysts.

36. An electrographic image development device, comprising the electrophotographic imaging member according to claim 35.
